(19) United States
(12) Patent Application Publication
(10) Pub. No.: US 2026/0060969 A1
(43) Pub. Date: Mar. 5, 2026

(54) METHODS FOR THE TREATMENT OF LYMPHOPROLIFERATIVE DISORDERS

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); CENTRE NATIONAL DE LA RECHERCHE SCIENTIFIQUE, Paris (FR); UNIVERSITÉ PARIS CITÉ, Paris (FR)

(72) Inventor: Guillaume CANAUD, PARIS (FR)

(21) Appl. No.: 19/101,058

(22) PCT Filed: Aug. 3, 2023
(86) PCT No.: PCT/EP2023/071533

§ 371 Date: Feb. 4, 2025

(30) Foreign Application Priority Data
Aug. 4, 2022 (EP) 22306188.8

(51) Int. Cl. A61K 31/4439; A61K 31/427; A61K 31/5377; A61K 31/553; A61K 45/06; A61P 35/00

(52) U.S. Cl. CPC A61K 31/4439; A61K 31/427; A61K 31/5377; A61K 31/553; A61K 45/06; A61P 35/00

(57) ABSTRACT

Inventors have first investigated the impact of PIK3CA inhibition in NZBWF1/J mice a model of lymphoproliferative disorders. They randomly assigned 30 females aged of 24 weeks to receive either vehicle (n=15) or alpelisib (n=15) during 4 weeks. At the time of sacrifice, alpelisib treated mice demonstrated significantly reduced spleen size. Flow cytometry analysis revealed that B cells were significantly reduced in alpelisib treated mice and CD8 cells count corrected. They then decided to explore the relevance of alpelisib in MRL/MpJ-Faslpr/J mice (referred here as MRL-lpr), another mouse model of lymphoproliferative disorder. These mice with homozygous Fas mutation usually develop severe lymphadenoproliferation. At the time of sacrifice. MRL-lpr mice treated with alpelisib demonstrated a reduction on their spleen and lymph node sizes. Flow cytometry analysis showed correction of B cells. T cells and other immune cells in peripheral blood mononuclear cells (PBMC), lymph nodes and spleen. The invention relates to a method for treating lymphoproliferative disorder in a subject in need thereof comprising a step of administering the subject with a therapeutically effective amount of a PIK3CA inhibitor.

### FIELD OF THE INVENTION

The invention relates to method and compositions for the treatment of lymphoproliferative disorders, such as B-cell lymphomas or T-cell lymphomas.

### BACKGROUND OF THE INVENTION

PIK3CA is a ubiquitously expressed lipid kinase that controls signaling pathways participating in cell proliferation, motility, survival and metabolism¹. PIK3CA is mainly recruited through tyrosine kinase receptors. PIK3CA encodes the 110-kDa catalytic alpha subunit of PI3K (p110α), which converts, at the plasma membrane, phosphatidylinositol 4,5-bisphosphate (PtdIns(4,5)P2) to phosphatidylinositol 3,4,5-trisphosphate (PtdIns(3,4,5)P3; or PIP3) with subsequent recruitment of PDK1, which in turn phosphorylates AKT on the Thr308 residue to initiate downstream cellular effects. PIK3CA also regulates many other pathways, including the Rho/Rac1 signaling cascade².

Due to the wide variability of clinical presentation and the difficulty of genetic identification, which most often requires a biopsy of the affected area, the exact prevalence of PIK3CA gain-of-function mutations is yet unknown. We recently, created a mouse model that recapitulates PIK3CA-Related Overgrowth Syndrome (PROS) patient phenotype. We identified alpelisib, a PIK3CA inhibitor undergoing development in oncology as a promising therapeutic in the mouse model and were authorized to treat PROS patients in poor condition using this drug. Patients treated with alpelisib demonstrated clinical, biological and radiological improvements.

In B cells, the PI3K pathway is recruited through the BCR. PI3Kδ is a heterodimeric enzyme, typically composed of a p85α regulatory subunit and a p110δ catalytic subunit. In B cells, PI3Kδ is activated upon cross-linking of the BCR, after stimulation with IL-4 or by the chemokine CXCL13 via CXCR5. The BCR co-opts the co-receptor CD19 or the adapter protein BCAP, both of which have YXXM motifs to which the p85α SH2 domains can bind. The IL-4R co-opts IRS1, which also has YXXM motifs. The mechanism whereby CXCR5 is coupled to PI3Kδ remains to be defined (indicated by a dotted line). PI3Kδ signalling through AKT promotes the activation of mTOR and suppresses FOXO1 function (via phosphorylation-dependent nuclear export). FOXO1 is a transcription factor that activates the genes encoding RAG proteins involved in V(D)J recombination, IKAROS which is required for early B cell development, CD62L which is required for homing to lymph nodes and AID, which is required for CSR and SHM. The amino acid sensor mTOR contributes to the growth and proliferation of B cells. All proteins coloured in green have been affected by LOF mutations causing PID. Of these, only p85α and p1108 have also been affected by GOF mutations causing APDS.

In T cells, the PI3K pathway is recruited through the TCR. PI3Kδ is a heterodimeric enzyme, typically composed of a p85α regulatory subunit and a p110δ catalytic subunit. In T cells, the TCR, the costimulatory receptor ICOS and the IL-2R can activate PI3Kδ. ICOS contains a YXXM motif in the cytoplasmic domain which is essential for ICOS-mediated co-stimulation. Precisely how the TCR activates PI3Kδ remains incompletely understood, though TCR ligation is known to induce ZAP70-mediated phosphorylation of LAT.

Whether PI3K binds LAT directly or via other adapter proteins remains to be established. Mechanisms of PI3Kδ activation downstream of IL-2R are even less clear, but a role for JAK3 has been implicated. PI3Kδ contributes to the downregulation of the expression of IL-7Rα and CD62L, via the AKT-dependent inactivation and nuclear export of FOXO1, preparing the T cell to exit the lymph nodes and circulate through the vascular systems and organs. PI3Kδ also increases metabolism and contributes to T cell effector-associated phenotypes by promoting activation of mTOR.

Thus, there is a need to understand mechanisms and find new therapeutic strategy to treat lymphoproliferative disorders.

### SUMMARY OF THE INVENTION

The present invention relates to a method for treating lymphoproliferative disorders in a subject in need thereof comprising a step of administering the subject with a therapeutically effective amount of a PIK3CA inhibitor. In particular, the present invention is defined by the claims.

### DETAILED DESCRIPTION OF THE INVENTION

Inventors have first investigated the impact of PIK3CA inhibition in NZBWF1/J mice a model of lymphoproliferative disorders. They randomly assigned 30 females aged of 24 weeks to receive either vehicle (n=15) or alpelisib (n=15) during 4 weeks. At the end of the treatment period, mice were sacrificed and flow cytometry was performed in splenocytes. At the time of sacrifice, alpelisib treated mice demonstrated significantly reduced spleen size. Flow cytometry analysis revealed that B cells were significantly reduced in alpelisib treated mice and CD8 cells count corrected.

They then decided to explore the relevance of alpelisib in MRL/MpJ-Faslpr/J mice (referred here as MRL-lpr), another mouse model of lymphoproliferative disorder. These mice with homozygous Fas mutation usually develop severe lymphadenoproliferation. Female mice die at an average of 18-20 weeks old. They first randomly assigned to either vehicle or alpelisib MRL-lpr female mice at 8 weeks old, before they fully develop diseases. At the time of sacrifice, MRL-lpr mice treated with alpelisib demonstrated a reduction on their spleen and lymph node sizes. Flow cytometry analysis showed correction of B cells, T cells and other immune cells in peripheral blood mononuclear cells (PBMC), lymph nodes and spleen.

They concluded that alpelisib and more generally PIK3CA inhibition represent promising drugs for patients with lymphoproliferative disorders.

Accordingly, in a first aspect, the present invention relates to a method for treating lymphoproliferative disorder in a subject in need thereof comprising a step of administrating the subject with a therapeutically effective amount of PI3K inhibitor, in particular PIK3CA inhibitor.

In some embodiments, the present invention also relates to a method for treating lymphoproliferative disorder in a subject in need thereof, wherein the method consists essentially in a step of administrating the subject with a therapeutically effective amount of PIK3CA inhibitor.

In some embodiments, the present invention also relates to a method for treating lymphoproliferative disorder in a subject in need thereof, wherein the method consists in a step of administrating the subject with a therapeutically effective amount of PIK3CA inhibitor.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the term “lymphoproliferative disorder” (LPD) refers to a heterogeneous group of diseases characterized by uncontrolled production of lymphocytes that cause monoclonal lymphocytosis, lymphadenopathy and bone marrow infiltration. These diseases often occur in immunocompromised individuals. There are two subsets of lymphocytes: T and B cells that regenerate uncontrollably to produce immunoproliferative disorders, which are prone to immunodeficiency, a dysfunctional immune system, and lymphocyte dysregulation.

In a particular embodiment, the lymphoproliferative disorder is a B-cell lymphoproliferative disorder.

In a particular embodiment, the B-lymphoproliferative disorder is selected from the group consisting of but not limited to: Hodgkin's lymphoma, Diffuse large B-cell lymphoma, acute lymphocytic leukemia, lymphoid blastic phase Chrome Myeloid Leukemia, Chronic lymphocytic leukemia/Small lymphocytic lymphoma, Extranodal marginal zone B-cell lymphomas, Mucosa-associated lymphoid tissue lymphomas, Follicular lymphoma, Mantle cell lymphoma, Nodal marginal zone B-cell lymphoma, Burkitt lymphoma, Hairy cell leukemia, Primary central nervous system lymphoma, Splenic marginal zone B-cell lymphoma, Waldenstrom's macroglobulinemia/Lymphoplasmacytic lymphoma, Multiple myeloma, Plasma cells dyscrasias, Plasma cell neoplasms, Primary mediastinal B-cell lymphoma, Hodgkin Disease or Castelman's Disease.

In a particular embodiment, the lymphoproliferative disorder is a T-cell lymphoproliferative disorder.

In a particular embodiment, the T-lymphoproliferative disorder is selected from the group consisting of but not limited to: leukemia/lymphoma, Extranodal natural killer/T-cell lymphoma, Cutaneous T-cell lymphoma, Enteropathy-type T-cell lymphoma, Angioimmunoblastic T-cell lymphoma, Anaplastic large T/null-cell lymphoma, Subcutaneous panniculitis-like T-cell lymphoma, T-cell acute lymphocytic leukemia, T-cell large granular lymphocyte leukemia, Lymphoid blastic phase Chrome Myeloid Leukemia, post-transplantation lymphoproliferative syndromes, human T-cell leukemia virus type 1-positive (HTLV-G) adult T-cell leukemia/lymphoma (ATL), T-cell prolymphocytic leukemia (T-PLL), or unspecified T-cell lymphoma.

As used herein, the term “subject” refers to any mammals, such as a rodent, a feline, a canine, and a primate. Particularly, in the present invention, the subject is a human afflicted with or susceptible to be afflicted with at least one of disorder lymphoproliferative disorder as described above.

In a particular embodiment, the subject is a human afflicted with or susceptible to be afflicted with B-lymphoproliferative disorder.

In another embodiment, the subject is a human afflicted with or susceptible to be afflicted with T-lymphoproliferative disorder.

As used herein, the term “PI3K” refers to phosphoinositide 3-kinases also called phophatidylinositide 3-kinases. PI3K belongs to a family of enzymes which phosphorylate the 3′hydroxyl group of the inositol ring of the phosphatidylinositol (PtdIns). The PI3K signalling pathway can be activated, resulting in the synthesis of PIP3 from PIP2. PIK3CA is mainly recruited through tyrosine kinase receptors. PIK3CA encodes the 110-kDa catalytic alpha subunit of PI3K (p110α), which converts, at the plasma membrane, phosphatidylinositol 4,5-bisphosphate (PtdIns(4,5)P2) to phosphatidylinositol 3,4,5-trisphosphate (PtdIns(3,4,5)P3; or PIP3) with subsequent recruitment of PDK1, which in turn phosphorylates AKT on the Thr308 residue to initiate downstream cellular effects. PIK3CA also regulates many other pathways, including the Rho/Rac1 signaling cascade.

As used herein, the term “PIK3CA inhibitor” refers to a natural or synthetic compound that has a biological effect to inhibit the activity or the expression of PI3K. More particularly, such compound is capable of inhibiting the kinase activity of at least one member of PI3K family, for example, at least a member of Class I PI3K. In particular embodiment, said PI3K inhibitor may be a pan-inhibitor of Class I PI3K (known as p110) or isoform specific of Class I PI3K isoforms (among the four types of isoforms, p110α, p110β, p110γ or p110δ).

In a particular embodiment, the PI3K inhibitor is a peptide, peptidomimetic, small organic molecule, antibody, aptamers, siRNA or antisense oligonucleotide. The term “peptidomimetic” refers to a small protein-like chain designed to mimic a peptide. In a particular embodiment, the inhibitor of PI3K is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the PI3K inhibitor is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the PI3K inhibitor is a small molecule which is an isoform-selective inhibitor of PI3K selected among the following compounds: BYL719 (Alpelisib, Novartis), GDC-0032 (Taselisib, Genentech/Roche), BKM120 (Buparlisib), TAK-117/MLN1117/INK1117 (Serabelisib), A66 (University of Auckland—CAS No.: 1166227-08-2), GSK260301 (Glaxosmithkline), KIN-193 (Astra-Zeneca—CAS No.: 1173900-33-8), TGX221 (Monash University—CAS No.: 663619-89-4), TG-1202 (Umbralisib), CAL101 (Idelalisib, Gilead Sciences), GS-9820 (Acalisib, Gilead Sciences), AMG319 (Amgen—CAS No. 1608125-21-8), IC87114 (Icos Corporation—CAS No.: 371242-69-2), BAY80-6946 (Copanlisib, Bayer Healthcare), GDC0941 (Pictilisib, Genentech), IPI145 (Duvelisib, Infinity), SAR405 (Sanofi—CAS No. 1523406-39-4), PX-866 (Sonolisib, Oncothyreon), perifosine, BEZ235 (Dactolisib), CUDC-907 (Fimepinostat), SAR245409/XL765 (Voxtalisib), XL-147 (Pilaralisib), GDC-0077 (Inavolisib), AZD-8186 (Astra-Zeneca-CAS No. 1627494-13-6), IPI549 (Eganelisib) or their pharmaceutically acceptable salts. In a more particular embodiment, the isoform-selective inhibitor of PI3K is selected among the following compounds: BYL719 (Alpelisib, Novartis), A66 (University of Auckland), GDC-0077 (Inavolisib, Genentech/Roche), CYH33 (Risovalisib), TAK-117/MLN1117/INK1117 (Serabelisib) or their pharmaceutically acceptable salts.

In an even more particular embodiment, the isoform-selective inhibitor of PI3K is selected among the following compounds: BYL719 (Alpelisib, Novartis), GDC-0077 (Inavolisib, Genentech/Roche), TAK-117/MLN1117/INK1117 (Serabelisib) or their pharmaceutically acceptable salts.

Such PI3K inhibitors are well-known in the art and described for example in Wang et al Acta Pharmacological Sinica (2015) 36: 1170-1176.

In a particular embodiment, the PI3K inhibitor is BYL719 and its derivatives.

As used herein, the term “BYL719” also called alpelisib is an ATP-competitive oral PI3K inhibitor selective for the p110α isoform that is activated by a mutant PIK3CA gene (Furet P., et al. 2013; Fritsch C., et al 2014). This molecule is also called Alpelisib and has the following formula and structure in the art C₁₉H₂₂F₃N₅O₂S:

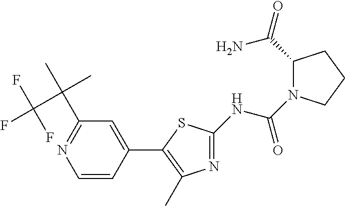 [SMILES: Cc1nc(NC(=O)N2CCC[C@H]2C(N)=O)sc1-c1ccnc(C(C)(C)C(F)(F)F)c1]

In a particular embodiment, the PI3K inhibitor is GDC-0032 and its derivatives, developed by Roche. This molecule also called Taselisib has the following formula and structure in the art C₂₄H₂₈N₈O₂:

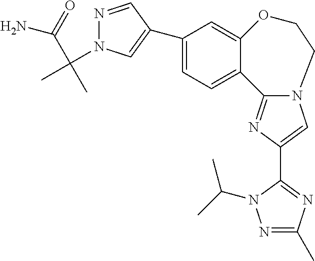 [SMILES: Cc1nc(-c2cn3c(n2)-c2ccc(-c4cnn(C(C)(C)C(N)=O)c4)cc2OCC3)n(C(C)C)n1]

In some embodiments, the PI3K inhibitor is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388. In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the PI3K inhibitor is an intrabody having specificity for PI3K. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the PI3K inhibitor is a short hairpin RNA (shRNA), a small interfering RNA (siRNA) or an antisense oligonucleotide which inhibits the expression of USP14. In a particular embodiment, the inhibitor of USP14 expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PI3K expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339:823-826), Xenopus tropicalis (Guo et al., 2014, Development, Vol. 141:707-714.), yeast (DiCarlo et al., 2013, Nucleic Acids Res., Vol. 41:4336-4343.), Drosophila (Gratz et al., 2014 Genetics, doi:10.1534/genetics.113.160713), monkeys (Niu et al., 2014, Cell, Vol. 156:836-843.), rabbits (Yang et al., 2014, J. Mol. Cell Biol., Vol. 6:97-99.), pigs (Hai et al., 2014, Cell Res. doi: 10.1038/cr.2014.11.), rats (Ma et al., 2014, Cell Res., Vol. 24:122-125.) and mice (Mashiko et al., 2014, Dev. Growth Differ. Vol. 56:122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Prevotella and Francisella 1 (Cpf1) in Zetsche et al. (“Cpf1 is a Single RNA-guided Endonuclease of a Class 2 CRISPR-Cas System (2015); Cell; 163, 1-13).

In a second aspect, the invention relates to the PIK3CA inhibitor for use according to the invention, and a classical treatment as a combined preparation for simultaneous, separate or sequential use in the treatment of lymphoproliferative disorder in a subject in need thereof.

As used herein, the terms “combined treatment”, “combined therapy” or “therapy combination” refer to a treatment that uses more than one medication. The combined therapy may be dual therapy or bi-therapy.

As used herein, the term “classical treatment” refers to treatments well known in the art and used to treat lymphoproliferative disorder (Hahn et al 2013, Arthritis Care Res (Hoboken). 2012 June; 64(6): 797-808; doi: 10.1002/acr.21664).

In the context of the invention, the classical treatment is selected from the group consisting of but not limited to: intravenous immunoglobulins (IVIG), immunosuppressor, corticosteroids, glucocorticoid, MAPK, PAK, mTOR, TKI, PARP, EGFR and/or IMPDH inhibitors, rituximab and monoclonal antibodies against T and/or B cells, chemotherapy, total body or localized radiation.

When several inhibitors are used, a mixture of inhibitors is obtained. In the case of multi-therapy (for example, bi-, tri- or quadritherapy), at least one another inhibitor can accompany the PI3K inhibitor.

In another embodiment, the invention relates to a combination comprising a PI3K inhibitor, and intravenous immune globulin (“IVIG”).

As used herein, the term “intravenous immune globulin” refers to a product made up of antibodies that can be given intravenously (through a vein).

In another embodiment, the invention relates to a combination comprising a PIK3CA inhibitor and at least a monoclonal antibody against T and/or B cells.

In a particular embodiment, the combination according to the invention, wherein the monoclonal antibody against T and/or B cells is adalimumab, certolizumab pegol, golimumab, infliximab, bevacizumab, blinatumomab, bivolumab, tocilizumab, etanercept or magrolimab.

In a particular embodiment, the combination according to the invention, wherein the monoclonal antibody against T and/or B cells is rituximab.

As used herein the term “rituximab” refers to a chimeric monoclonal antibody targeted against CD20 which is a surface antigen present on B cells. In a particular embodiment, the PI3K inhibitor as described above is combined with an immunosuppressive therapy.

As used herein, the term “immunosuppressive therapy” refers to immunosuppressive treatment, which means that the subject is administered with one or more immunosuppressive drugs. Immunosuppressive drugs that may be employed in transplantation procedures include azathioprine (AZA), methotrexate, cyclophosphamide (CYC), FK-506 (tacrolimus), rapamycin, corticosteroids, and cyclosporin. These drugs may be used in monotherapy or in combination therapies.

In a particular embodiment, the immunosuppressive treatment is performed with azathioprine.

In a particular embodiment, the immunosuppressive treatment is performed with cyclophosphamide.

In another embodiment, the PI3K inhibitor as described above is combined with glucocorticoids therapy.

As used herein, the term “glucocorticoids therapy” refers to a class of corticosteroids, which are a class of steroid hormones. Glucocorticoids are corticosteroids that bind to the glucocorticoid receptor.

In a particular embodiment, the glucocorticoid therapy is performed with prednisone.

In another embodiment, the classical treatment is mycophenolate mofetil (MMF, CELLCEPT).

In a particular embodiment, the PI3K inhibitor, an immunosuppressor and a glucocorticoid can be combined as a tri-therapy for use in the treatment of lymphoproliferative disorder.

In a particular embodiment, the PI3K inhibitor, an immunosuppressor and a glucocorticoid can be combined as a tri-therapy, wherein the PI3K inhibitor, immunosuppressor and a glucocorticoid are BYL719, azathioprine or clophosphamide and prednisone respectfully.

In a particular embodiment, the PI3K inhibitor for use according to the invention, and an immunosuppressor, glucocorticoids, MAPK, PAK, mTOR, TK, PARP or EGFR inhibitor as a combined preparation for simultaneous, separate or sequential use in the treatment of lymphoproliferative disorder in a subject in need thereof.

In a particular embodiment, the PI3K inhibitor for use according to the invention, and an immunosuppressor, glucocorticoids, MAPK, PAK, mTOR, TK, PARP or EGFR inhibitor as a combined preparation for simultaneous, separate or sequential use in the treatment of lymphoproliferative disorder in a subject in need thereof.

In another embodiment, the invention relates to a combination comprising a PI3K inhibitor, and at least one classical treatment selected from the group consisting of immunosuppressor, glucocorticoids, MAPK, PAK, mTOR, TK, PARP or EGFR inhibitors as described below for use in the treatment of lymphoproliferative disorder in a subject in need thereof.

In another embodiment, the PI3K, MAPK and PAK inhibitors can be combined as a tri-therapy for use in the treatment of lymphoproliferative disorder. In a particular embodiment, the PI3K, MAPK and PAK inhibitors can be combined as a tri-therapy, wherein the PI3K, MAPK and inhibitors are BYL719, selumetinib and IPA-3 respectfully.

In a particular embodiment, the method according to the invention, wherein the PI3K inhibitor and a MAPK inhibitor, a PAK inhibitor, an mTOR inhibitor, a TKI, a PARP inhibitor or an EGFR inhibitor, as combined preparation for use simultaneously, separately or sequentially in the treatment of lymphoproliferative disorder.

As used herein, the term “MAPK” refers to mitogen-activated protein kinase, is a type of protein kinase that is specific to the amino acids serine and threonine. MAPK are involved in cellular responses to a diverse array of stimuli, such as mitogens, osmotic stress, heat shock and proinflammatory cytokines. Six groups of MAPK have so far been identified: Extracellular signal-regulated kinases (ERK1, ERK2), c-Jun N-terminal kinases (JNKs), p38 isoforms (MAPK11, MAPK12, MAPK13, MAPK14), ERK5 (MAPK7), ERK3 (MAPK6) and ERK4 (MAPK4), ERK7/8 (MAPK15). In a particular embodiment, the inhibitors of MAPK are inhibitors of ERK1/ERK2. The inhibitor of ERK1/ERK2 is selected from the group but is not limited to VTX-11e, SCH772984.

In a particular embodiment, the MAPK inhibitor is a peptide, peptidomimetic, small organic molecule, antibody, aptamers, siRNA or antisense oligonucleotide. In a particular embodiment, the MAPK inhibitor is p38-MAPK inhibitor. Typically, the inhibitor of p38-MAPK is selected from the group consisting of SB 203580, SB 203580 hydrochloride, SB681323 (Dilmapimod), LY2228820 dimesylate, BIRB 796 (Doramapimod), BMS-582949, Pamapimod, GW856553, ARRY-797AL 8697, AMG 548, CMPD-1, EO 1428, JX 401, RWJ 67657, TA 01, TA 02, VX 745,DBM 1285 dihydrochloride, ML 3403, SB 202190, SB 239063, SB 706504, SCIO 469 hydrochloride, SKF 86002 dihydrochloride, SX 011, TAK 715, VX 702, or PH-797804.

In a particular embodiment, the inhibitor of MAPK is an inhibitor of MEK. MEK1 and MEK2 are members of a larger family of dual-specificity kinases (MEK1-7) that phosphorylate threonine and tyrosine residues of various MAP kinases. In a particular embodiment, the inhibitor of MAPK is selected from the group consisting of Trametinib (GSK1120212); Selumetinib (AZD6244).

In a particular embodiment, the PI3K inhibitor for use according to the invention and, a MAPK inhibitor, as a combined preparation for simultaneous, separate or sequential use in the treatment of lymphoproliferative disorder in a subject in need thereof, wherein the PI3K inhibitor is BYL719 and, the MAPK inhibitor is Selumetinib.

In another embodiment, the PI3K inhibitor for use according to the invention, and PAK inhibitor as a combined preparation for simultaneous, separate or sequential use in the treatment of lymphoproliferative disorder in a subject in need thereof.

As used herein, the term “PAK” refers to p21-activated kinase which regulates cytoskeleton remodeling, phenotypic signaling and gene expression, and affects a wide variety of cellular processes such as directional motility, invasion, metastasis, growth, cell cycle progression, angiogenesis. In a particular embodiment, the PAK inhibitor is a peptide, peptidomimetic, small organic molecule, antibody, aptamers, siRNA or antisense oligonucleotide.

In a particular embodiment, the inhibitor of PAK is selected from the group consisting of PP1, hPIP1, NESH, Merlin, CRIPak, LKB1, Mesalamine, Glaucarubinone, Myricetin, β-elemene, miR-7, miR-let-7, miR-145, FRAX1036, OSU-03012, and IPA-3.

In a particular embodiment, the PAK inhibitor is used with thalidomide, lenalidomide or pomalidomide, as a combined preparation for use in the treatment of lymphoproliferative disorder.

In a particular embodiment, the PI3K inhibitor for use according to the invention and, a PAK inhibitor, as a combined preparation for simultaneous, separate or sequential use in the treatment of lymphoproliferative disorder in a subject in need thereof, wherein the PI3K inhibitor is BYL719 and the PAK inhibitor is IPA-3.

In another embodiment, the PI3K inhibitor for use according to the invention, and mTOR inhibitor as a combined preparation for simultaneous, separate or sequential use in the treatment of lymphoproliferative disorder in a subject in need thereof.

As used herein, the term “mTOR” refers to mammalian target of rapamycin also known as mechanistic target of rapamycin and FK506-binding protein 12-rapamycin-associated protein 1 (FRAP1). mTOR functions as a serine/threonine protein kinase that regulates cell growth, cell proliferation, cell motility, cell survival, protein synthesis, autophagy, and transcription. mTOR has two structurally distinct complexes: mTORC1 and mTORC2. In a particular embodiment, the mTOR inhibitor is a peptide, peptidomimetic, small organic molecule, antibody, aptamers, siRNA or antisense oligonucleotide.

In a particular embodiment, the inhibitor of mTOR is selected from the group consisting of rapamycin (also called sirolimus and described in U.S. Pat. No. 3,929,992), temsirolimus, deforolimus, everolimus, tacrolimus and rapamycin analogue or derivative thereof, AMG954, AZD8055, AZD2014, BEZ235, BGT226, CC-115, CC-223, LY3023414, P7170, DS-7423, OSI-027, GSK2126458, PF-04691502, PF-05212384, INK128, MLN0128, MLN1117, Ridaforolimus, Metformin, XL765, SAR245409, SF1126, VS5584, GDC0980 and GSK2126458.

As used herein, the term “rapamycin analogue or derivative thereof” includes compounds having the rapamycin core structure as defined in U.S. Patent Application Publication No. 2003/0008923 (which is herein incorporated by reference), which may be chemically or biologically modified while still retaining mTOR inhibiting properties. Such derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus.

Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462).

Examples of 27-esters and ethers of rapamycin are disclosed in U.S. Pat. No. 5,256,790, which is hereby incorporated by reference in its entirety.

Examples of oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference.

Other compounds within the scope of “rapamycin analog or derivative thereof” include those compounds and classes of compounds referred to as “rapalogs” in, for example, WO 98/02441 and references cited therein, and “epirapalogs” in, for example, WO 01/14387 and references cited therein.

Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD. Another preferred mTOR inhibitor is zotarolimus, an antiproliferative agent (Abbott Laboratories). Zotarolimus is believed to inhibit smooth muscle cell proliferation with a cytostatic effect resulting from the inhibition of mTOR. Another preferred mTOR inhibitor is tacrolimus, a macrolide lactone immunosuppressant isolated from the soil fungus Streptomyces tsukubaensis. Tacrolimus is also known as FK 506, FR 900506, Fujimycin, L 679934, Tsukubaenolide, PROTOPIC and PROGRAF. Other preferred mTOR inhibitors include AP-23675, AP-23573, and AP-23841 (Ariad Pharmaceuticals).

Preferred rapamycin derivatives include everolimus, CCI-779 (rapamycin 42-ester with 3-hydroxy-2-(hydroxymethyl)-2-methylpropionic acid; U.S. Pat. No. 5,362,718); 7-epi-rapamycin; 7-thiomethyl-rapamycin; 7-epi-trimethoxyphenyl-rapamycin; 7-epi-thiomethyl-rapamycin; 7-demethoxy-rapamycin; 32-demethoxy-rapamycin; 2-desmethyl-rapamycin; and 42-O-(2-hydroxy)ethyl rapamycin (U.S. Pat. No. 5,665,772).

Additional mTORC2 inhibitors may be OSI-027 (OSI Pharmaceuticals), a small molecule mTORC2 inhibitor. OSI-027 inhibits mTORC2 signaling complexes, allowing for the potential for complete truncation of aberrant cell signaling through this pathway.

In addition, torkinibs, ATP-competitive mTOR kinase domain inhibitors and inhibitors of mTORC2 may also be used according to the invention. Exemplary torkinibs include PP242 and PP30 (see, Feldman et al. (2009) PLoS Biology 7:371) and Torin1 (Thoreen et al. (2009) J Biol Chem 284:8023).

In a particular embodiment, the PI3K inhibitor for use according to the invention and, a mTOR inhibitor, as a combined preparation for simultaneous, separate or sequential use in the treatment of lymphoproliferative disorder in a subject in need thereof, wherein the PI3K inhibitor is BYL719 and the mTOR inhibitor is everolimus.

In another embodiment, the PI3K inhibitor for use according to the invention, and tyrosine kinase inhibitor (TKI) as a combined preparation for simultaneous, separate or sequential use in the treatment of lymphoproliferative disorder in a subject in need thereof.

As used herein, the term “TKI” refers to tyrosine kinase inhibitor. Tyrosine kinase is involved in the phosphorylation of many proteins. Example of tyrosine kinase proteins: AATK; ABL; ABL2; ALK; AXL; BLK; BMX; BTK; CSF1R; CSK; DDR1; DDR2; EGFR; EPHA1; EPHA2; EPHA3; EPHA4; EPHA5; EPHA6; EPHA7; EPHA8; EPHA10; EPHB1; EPHB2; EPHB3; EPHB4; EPHB6; ERBB2; ERBB3; ERBB4; FER; FES; FGFR1; FGFR2; FGFR3; FGFR4; FGR; FLT1; FLT3; FLT4; FRK; FYN; GSG2; HCK; IGF1R; ILK; INSR; INSRR; IRAK4; ITK; JAK1; JAK2; JAK3; KDR; KIT; KSR1; LCK; LMTK2; LMTK3; LTK; LYN; MATK; MERTK; MET; MLTK; MST1R; MUSK; NPR1; NTRK1; NTRK2; NTRK3; PDGFRA; PDGFRB; PLK4; PTK2; PTK2B; PTK6; PTK7; RET; ROR1; ROR2; ROS1; RYK; SGK493; SRC; SRMS; STYK1; SYK; TEC; TEK; TEX14; TIE1; TNK1; TNK2; TNNI3K; TXK; TYK2; TYRO3; YES1; ZAP70. In a particular embodiment, the TKI is a peptide, peptidomimetic, small organic molecule, antibody, aptamers, siRNA or antisense oligonucleotide.

In a particular embodiment, the tyrosine kinase is EGFR. As used herein, the term “EGFR” refers to epidermal growth factor receptor which is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). EGFR are involved in the differentiation and cell growth. Inhibitors of EGFR refer to compounds which inhibits cell growth. In a particular embodiment, the inhibitor of EGFR is selected from the group consisting of: gefitinib, erlotinib, afatinib, brigatinib, lapatinib, icotinib, cetuximab Osimertinib, zalutumumab, nimotuzumab, and matuzumab.

In a particular embodiment, the inhibitor of EGFR is an irreversible mutant-selective EGFR inhibitor that specifically targets EGFR-activating mutations arising de novo and upon resistance acquisition. Typically, such inhibitor inhibits the most common EGFR mutations L858R, Ex19del, and T790M. Accordingly, in a particular embodiment, the inhibitor of EGFR is EGF816 also known as Nazartinib developed by Novartis.

In a particular embodiment, the tyrosine kinase is VEGF. As used herein, the term “VEGF” refers to vascular endothelial growth factor. VEGF is involved in stimulate cellular responses by binding to tyrosine kinase receptors (the VEGFRs) on the cell surface, notably to stimulate the formation of blood vessel (angiogenesis). VEGF family comprises in mammals five members: VEGF-A, placenta growth factor (PGF), VEGF-B, VEGF-C and VEGF-D. In a particular embodiment, the inhibitors of VEGF refer to inhibit the stimulation of growth cells and formation of blood vessel. In a particular embodiment, the inhibitor of VEGF is selected from the group consisting of: ranibizumab (Lucentis®), aflibercept (Eylea®) and bevacizumab (Avastin®), Tivozanib, Lenvatinib, Axitinib, Imtinib, or brolucizumab (RTH258).

In another embodiment, the inhibitor is a VEGFR inhibitor. As used herein, the term “VEGFR” refers to receptors for vascular endothelial growth factor (VEGF). Three main subtypes of VEGFR exist: VEGFR1, VEGFR 2 and VEGFR 3. VEGFR inhibitor is selected from the group consisting of: Pegaptanib, lenvatinib, motesanib, Pazopanib, cabozantinib (Cabometyx®).

In some embodiments, the TKI is selected from the group consisting of gefitinib, erlotinib, dasatinib, nilotinib, bosutinib, ponatinib, ruxolitinib, quizartinib, cabozantinib and sunitinib. In a specific embodiment, the TKI is imatinib.

In another embodiment, the PI3K inhibitor for use according to the invention, and PARP inhibitor as a combined preparation for simultaneous, separate or sequential use in the treatment of lymphoproliferative disorder in a subject in need thereof.

As used herein, the term “PARP” refers to Poly (ADP-ribose) polymerase which is an enzyme involved in cellular processes such as DNA repair, genomic stability, and programmed cell death. In a particular embodiment, the PARP inhibitor is a peptide, peptidomimetic, small organic molecule, antibody, aptamers, siRNA or antisense oligonucleotide.

The PARP inhibitor is selected from the group consisting of: iniparib (BSI 201), talazoparib (also known as BMN-673), velipari (ABT-888), olaparib (also known as AZD-2281 and commercialized as Lynparza®), rucaparib (also known as Rubraca®) or niraparib (also known as Zejula®).

The PI3K, MAPK, PAK, mTOR, TKI, PARP and/or EGFR inhibitors as described above can be used as part of a multi-therapy for the treatment of lymphoproliferative disorder in a subject in need thereof.

The PI3K inhibitor can be used alone as a single inhibitor or in combination with other inhibitors like MAPK, PAK, mTOR, TKI, PARP and/or EGFR inhibitors. When several inhibitors are used, a mixture of inhibitors is obtained. In the case of multi-therapy (for example, bi-, tri- or quadritherapy), at least one other inhibitor can accompany the PI3K inhibitor.

In a particular embodiment, the PI3K and MAPK inhibitors can be combined as a bi-therapy for use in the treatment of lymphoproliferative disorder. In a particular embodiment, the PI3K and MAPK inhibitors can be combined for use as a bi-therapy, wherein the PI3K and MAPK inhibitors are BYL719 and selumetinib respectfully.

In another embodiment, the PI3K and ERK inhibitors can be combined as a bi-therapy for use in the treatment lymphoproliferative disorder. In a particular embodiment, the PI3K and ERK inhibitors can be combined for use as a bi-therapy, wherein the PI3K and ERK inhibitors are BYL719 and VTX-11e respectfully.

In another embodiment, the PI3K and mTOR inhibitors can be combined as a bi-therapy for use in the treatment lymphoproliferative disorder. In a particular embodiment, the PI3K and mTOR inhibitors can be combined for use as a bi-therapy, wherein the PI3K and mTOR inhibitors are BYL719 and everolimus respectfully.

In another embodiment, the PI3K and TK inhibitors can be combined as a bi-therapy for use in the treatment lymphoproliferative disorder. In a particular embodiment, the PI3K and TK inhibitors can be combined for use as a bi-therapy, wherein the PI3K and TK inhibitors are BYL719 and sunitinib respectfully.

In another embodiment, the PI3K and VEGF inhibitors can be combined as a bi-therapy for use in the treatment lymphoproliferative disorder. In a particular embodiment, the PI3K and TK inhibitors can be combined for use as a bi-therapy, wherein the PI3K and VEGF inhibitors are BYL719 and brolucizumab (RTH258) respectfully.

In another embodiment, the PI3K, MAPK and PAK inhibitors can be combined as a tri-therapy for use in the treatment of lymphoproliferative disorder. In a particular embodiment, the PI3K, MAPK and PAK inhibitors can be combined as a tri-therapy, wherein the PI3K, MAPK and inhibitors are BYL719, selumetinib and IPA-3 respectfully.

In another embodiment, the PI3K and chemotherapy can be combined as a bi-therapy for use in the treatment lymphoproliferative disorder.

As used herein, the term “chemotherapy” refers to use of chemotherapeutic agents to treat a subject. As used herein, the term “chemotherapeutic agent” refers to chemical compounds that are effective in inhibiting tumor growth in lymphoproliferative disorder.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancrati statin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Inti. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2, 2′, 2″-trichlorotriethylamine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In another embodiment, the PI3K and radiotherapy can be combined as a bi-therapy for use in the treatment lymphoproliferative disorder.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein the terms “administering” or “administration” refer to the act of injecting or otherwise physically delivering a substance as it exists outside the body (e.g., an inhibitor of PI3K) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the term “administration simultaneously” refers to administration of at least 2 or 3 active ingredients by the same route and at the same time or at substantially the same time. The term “administration separately” refers to an administration of at least 2 or 3 active ingredients at the same time or at substantially the same time by different routes. The term “administration sequentially” refers to an administration of at least 2 or 3 active ingredients at different times, the administration route being identical or different.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The PIK3CA inhibitor alone or combined with a classical treatment, as described above may be combined with pharmaceutically acceptable excipients, and optionally sustained-release matrices, such as biodegradable polymers, to form pharmaceutical compositions.

Accordingly, in a third aspect, the invention relates to a pharmaceutical composition comprising a PIK3CA inhibitor for use in the treatment of lymphoproliferative disorder as described above.

In a particular embodiment, the invention relates to a pharmaceutical composition comprising a PIK3CA inhibitor for use in the treatment of B-lymphoproliferative disorder.

In a particular embodiment, the invention relates to a pharmaceutical composition comprising a PIK3CA inhibitor for use in the treatment of T-lymphoproliferative disorder.

In a further embodiment, the invention relates to a pharmaceutical composition comprising i) a PIK3CA inhibitor and ii) a classical treatment as described above as combined preparation to treat lymphoproliferative disorder.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

A further object of the present invention relates to a method of screening a drug suitable for the treatment of lymphoproliferative disorder comprising i) providing a test compound and ii) determining the ability of said test compound to inhibit the activity of PI3K.

Any biological assay well known in the art could be suitable for determining the ability of the test compound to inhibit the activity of PI3K. In some embodiments, the assay first comprises determining the ability of the test compound to bind to PI3K. In some embodiments, a population of cells is then contacted and activated so as to determine the ability of the test compound to inhibit the activity of PI3K. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of PI3K, as determined using in vitro methods described herein, are likely to exhibit similar modulatory capacity in applications in vivo. Typically, the test compound is selected from the group consisting of peptides, peptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: Alpelisib improves lymphoproliferative mouse models. A. Spleen to body weight ratio at the time of sacrifice (4 weeks following treatment either with vehicle or alpelisib) (n=5-15 mice per group). B-F. Percentage of spleen cell population determined by flow cytometry analysis in NZBWF1/J mice treated with either vehicle or alpelisib. Spleen to body weight ratio of MRL-lpr mice treated either with vehicle or alpelisib for 4 weeks (n=14 mice per group). Percentage of peripheral blood monuclear cell (PBMC), bone marrow, spleen and lymph nodes cell population determined by flow cytometry analysis in MRL-lpr mice treated with either vehicle or alpelisib for 4 weeks (n=8-9 mice per group). CD4, CD8, B220, Mac1, Gr1 are used as lineage markers.

FIG. 2: Alpelisib modifies mononuclear cell population in MRL-lpr mice. Flow cytometry analysis of bone marrow, spleen, lymph nodes, and PBMC in MRL-lpr mice treated either with vehicle or alpelisib for 4 weeks (n=8-9 mice per group). CD4, CD8, B220, Mac1, Gr1 are used as lineage markers.

### EXAMPLES

### Example 1

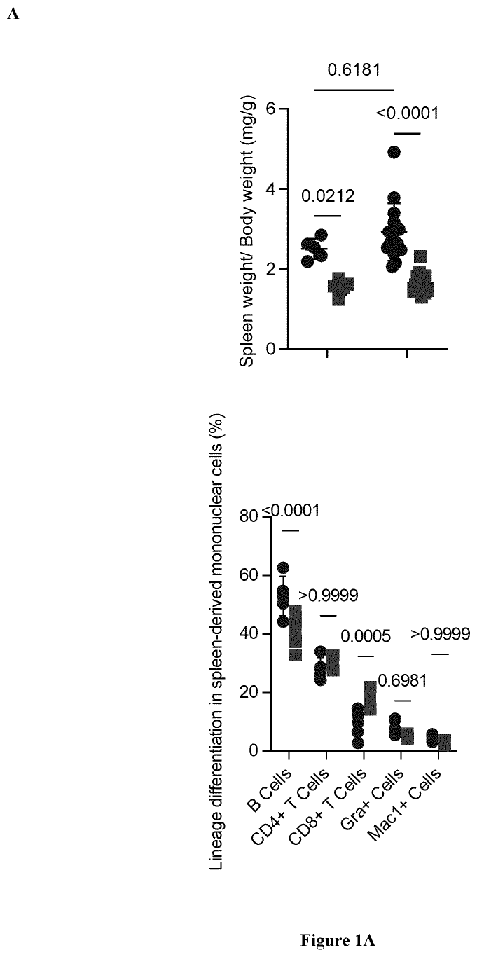

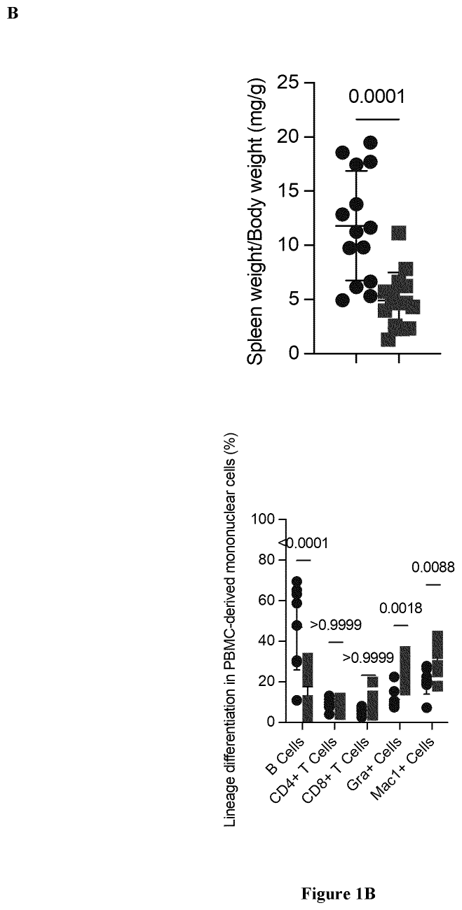

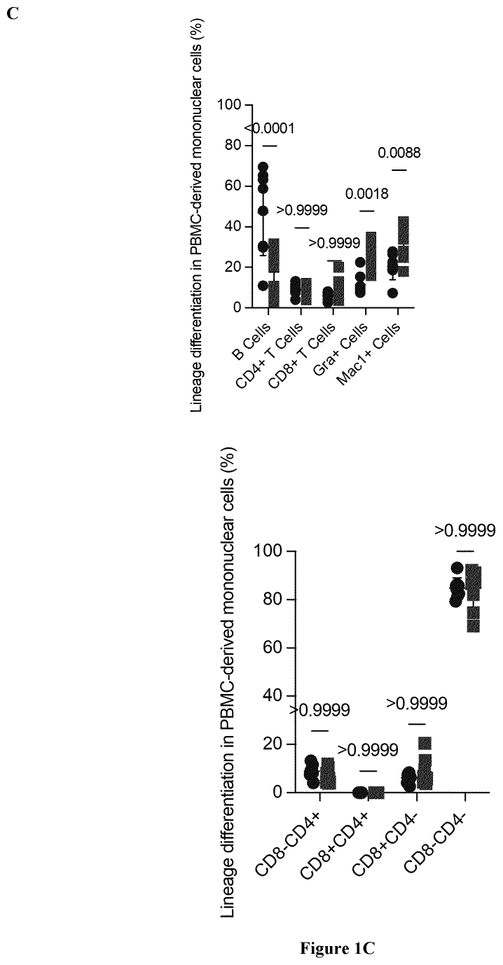

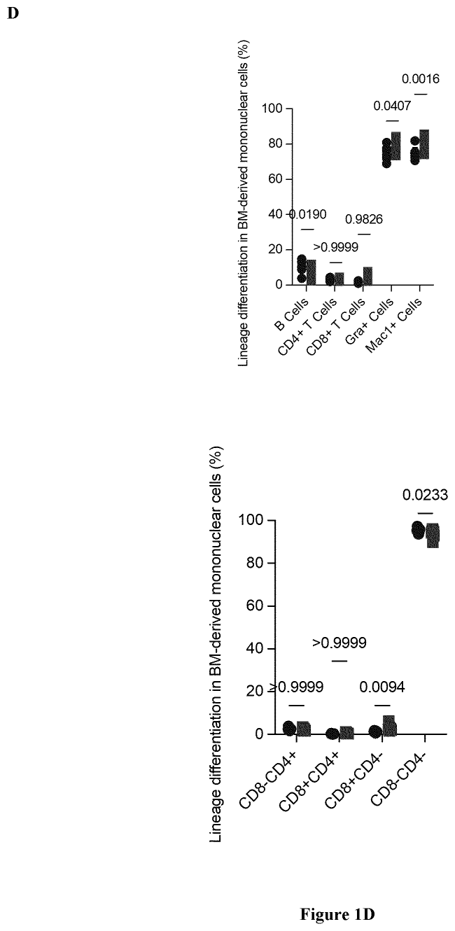

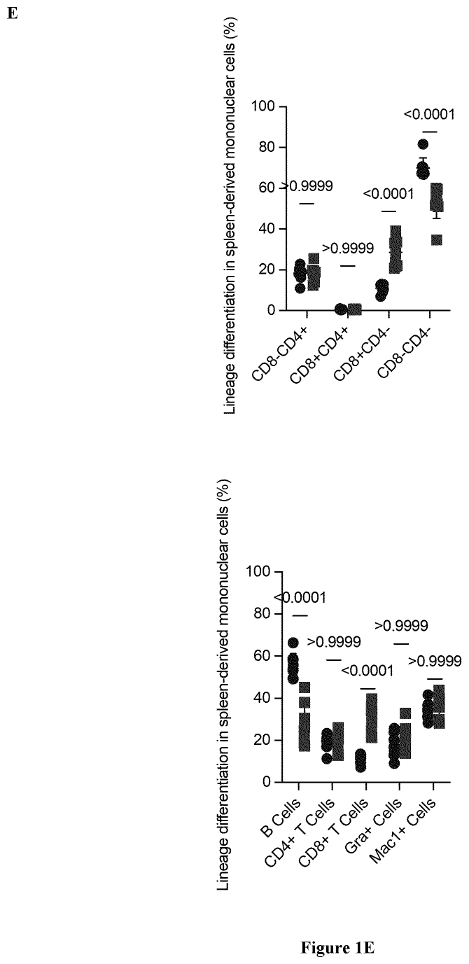

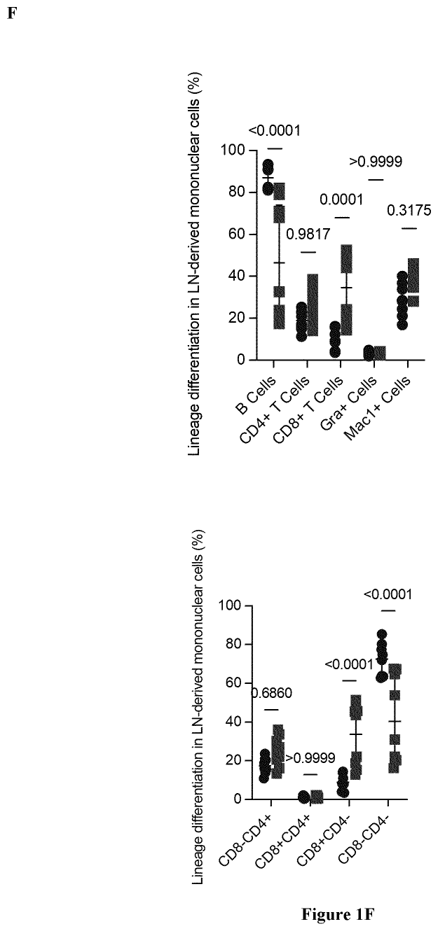

We first investigated the impact of PIK3CA inhibition in NZBWF1/J mice a model of lymphoproliferative disorders. We randomly assigned 30 females aged of 24 weeks to receive either vehicle (n=15) or alpelisib (n=15) during 4 weeks. At the end of the treatment period, mice were sacrificed and flow cytometry was performed in splenocytes. At the time of sacrifice, alpelisib treated mice demonstrated significantly reduced spleen size (FIG. 1A). Flow cytometry analysis revealed that B cells were significantly reduced in alpelisib treated mice and CD8 cells count corrected (FIG. 1B, C, D, E, F).

### Example 2

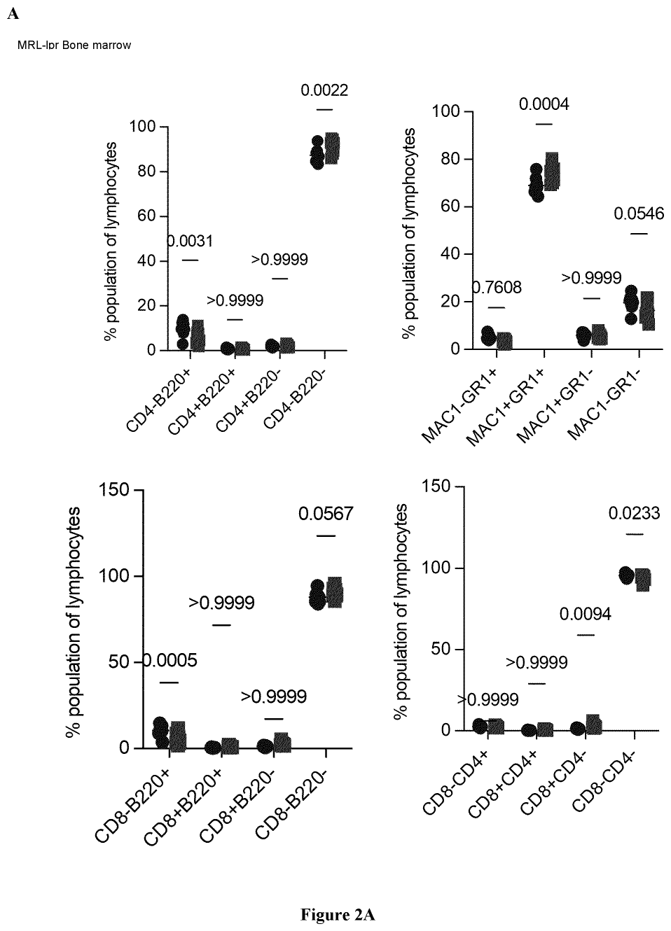

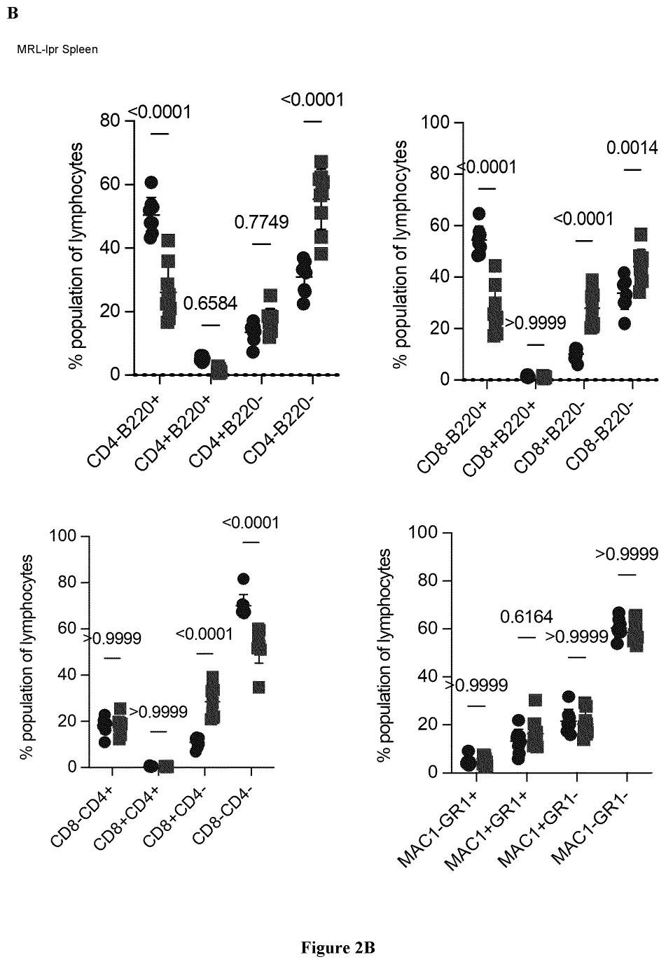

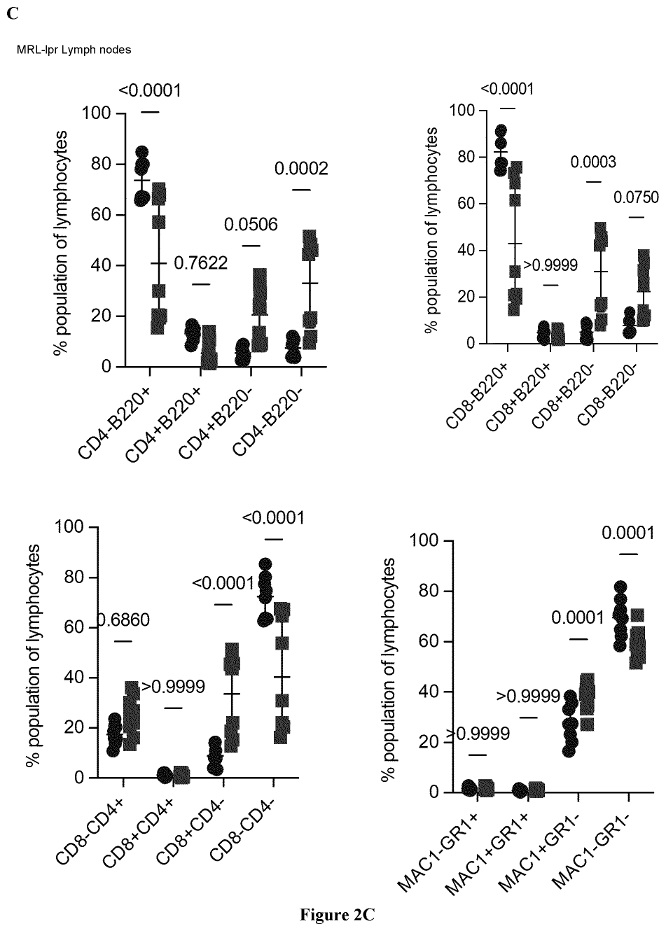

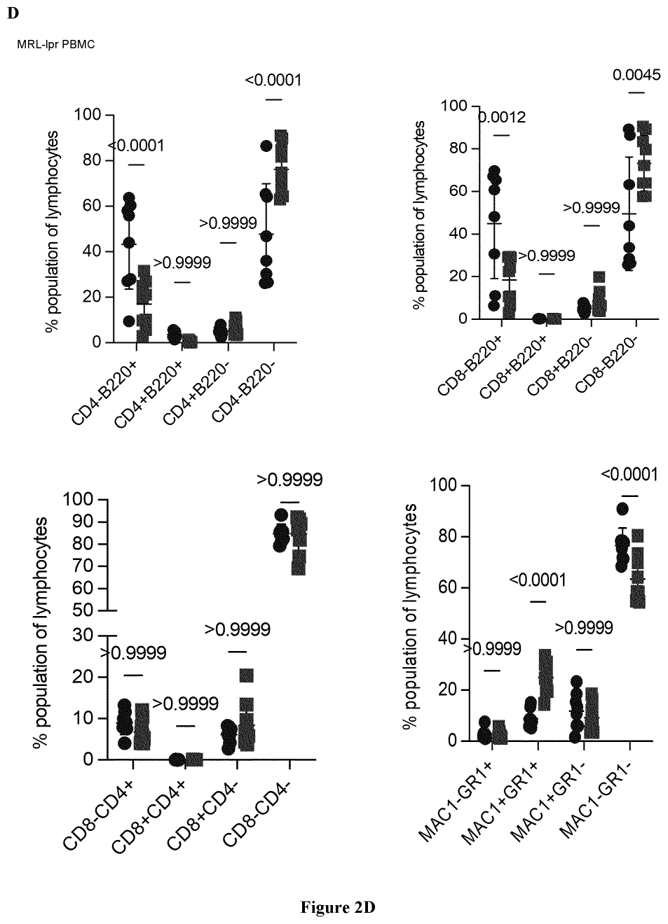

We then decided to explore the relevance of alpelisib in MRL/MpJ-Faslpr/J mice (referred here as MRL-lpr), another mouse model of lymphoproliferative disorder. These mice with homozygous Fas mutation usually develop severe lymphadenoproliferation. Female mice die at an average of 18-20 weeks old. We first randomly assigned to either vehicle or alpelisib MRL-lpr female mice at 8 weeks old, before they fully develop diseases. At the time of sacrifice, MRL-lpr mice treated with alpelisib demonstrated a reduction on their spleen and lymph node sizes (FIG. 1B, C, D, E, F). Flow cytometry analysis showed correction of B cells, T cells and other immune cells in peripheral blood mononuclear cells (PBMC), lymph nodes and spleen (FIG. 1B, C, D, E, F and FIG. 2A, B, C, D).

We concluded that alpelisib and more generally PIK3CA inhibition represent promising drugs for patients with lymphoproliferative disorders.

### REFERENCES

Throughout this application, various references describe the state of the art to which this invention pertains. The disclosures of these references are hereby incorporated by reference into the present disclosure.
1 Bilanges, B., Posor, Y. & Vanhaesebroeck, B. PI3K isoforms in cell signalling and vesicle trafficking. Nat Rev Mol Cell Biol, doi:10.1038/s41580-019-0129-z (2019).
2 Welch, H. C., Coadwell, W. J., Stephens, L. R. & Hawkins, P. T. Phosphoinositide 3-kinase-dependent activation of Rac. FEBS Lett 546, 93-97 (2003).
3 Kurek, K. C. et al. Somatic mosaic activating mutations in PIK3CA cause CLOVES syndrome. Am J Hum Genet 90, 1108-1115, doi:10.1016/j.ajhg.2012.05.006 (2012).
4 Keppler-Noreuil, K. M. et al. Clinical delineation and natural history of the PIK3CA-related J overgrowth spectrum. Am Med Genet A 164A, 1713-1733, doi:10.1002/ajmg.a.36552 (2014).
5 Keppler-Noreuil, K. M. et al. PIK3CA-related overgrowth spectrum (PROS): diagnostic and testing eligibility criteria, differential diagnosis, and evaluation. Am J Med Genet A 167A, 287-295, doi:10.1002/ajmg.a.36836 (2015).
6 Luks, V. L. et al. Lymphatic and other vascular malformative/overgrowth disorders are caused by somatic mutations in PIK3CA. J Pediatr 166, 1048-1054 e1041-1045, doi:10.1016/j.jpeds.2014.12.069 (2015).
7 Mirzaa, G. M. et al. Megalencephaly-capillary malformation (MCAP) and megalencephaly-polydactyly-polymicrogyria-hydrocephalus (MPPH) syndromes: two closely related disorders of brain overgrowth and abnormal brain and body morphogenesis. Am J Med Genet A 158A, 269-291, doi:10.1002/ajmg.a.34402 (2012).
8 Rios, J. J. et al. Somatic gain-of-function mutations in PIK3CA in patients with macrodactyly. Hum Mol Genet 22, 444-451, doi:10.1093/hmg/dds440 (2013).
9 Rivière, J. B. et al. De novo germline and postzygotic mutations in AKT3, PIK3R2 and PIK3CA cause a spectrum of related megalencephaly syndromes. Nat Genet 44, 934-940, doi:10.1038/ng.2331 (2012).

1. A method for treating a lymphoproliferative disorder in a subject in need thereof comprising administering to the subject a therapeutically effective amount of a PIK3CA inhibitor.

2. The method according to claim 1, wherein the method consists essentially of administrating to the subject a therapeutically effective amount of PIK3CA inhibitor.

3. The method according to claim 1, wherein the lymphoproliferative disorder is a B-cell lymphoproliferative disorder.

4. The method according to claim 3, wherein the B-lymphoproliferative disorder is selected from the group consisting of: Hodgkin's lymphoma, Diffuse large B-cell lymphoma, acute lymphocytic leukemia, lymphoid blastic phase Chrome Myeloid Leukemia, Chronic lymphocytic leukemia/Small lymphocytic lymphoma, Extranodal marginal zone B-cell lymphomas, Mucosa-associated lymphoid tissue lymphomas, Follicular lymphoma, Mantle cell lymphoma, Nodal marginal zone B-cell lymphoma, Burkitt lymphoma, Hairy cell leukemia, Primary central nervous system lymphoma, Splenic marginal zone B-cell lymphoma, Waldenstrom's macroglobulinemia/Lymphoplasmacytic lymphoma, Multiple myeloma, Plasma cells dyscrasias, Plasma cell neoplasms, Primary mediastinal B-cell lymphoma, Hodgkin Disease and Castelman's Disease.

5. The method according to claim 1, wherein the lymphoproliferative disorder is a T-cell lymphoproliferative disorder.

6. The method according to claim 5, wherein the T-lymphoproliferative disorder is selected from the group consisting of: leukemia/lymphoma, Extranodal natural killer/T-cell lymphoma, Cutaneous T-cell lymphoma, Enteropathy-type T-cell lymphoma, Angioimmunoblastic T-cell lymphoma, Anaplastic large T/null-cell lymphoma, Subcutaneous panniculitis-like T-cell lymphoma, T-cell acute lymphocytic leukemia, T-cell large granular lymphocyte leukemia, Lymphoid blastic phase Chrome Myeloid Leukemia, post-transplantation lymphoproliferative syndromes, human T-cell leukemia virus type 1-positive (HTLV-G) adult T-cell leukemia/lymphoma (ATL), T-cell prolymphocytic leukemia (T-PLL), and unspecified T-cell lymphoma

7. The method according to claim 1, wherein the PIK3CA inhibitor is selected from the group consisting of: BYL719, A66, GDC-0077, CYH33, TAK-117/MLN1117/INK1117 or a pharmaceutically acceptable salts thereof.

8. The method according to claim 1, wherein the PIK3CA inhibitor is BYL719 or a derivative thereof.

9. The method of claim 1, wherein the PIK3CA inhibitor is administered with a classical treatment.

10. The method according to claim 9, wherein the classical treatment is selected from the group consisting of: an intravenous immunoglobulin (IVIG), an immunosuppressor, a corticosteroid, a glucocorticoid, a mitogen-activated protein kinase (MAPK) inhibitor, a p21-activated kinase (PAK) inhibitor, an mTOR inhibitor, a tyrosine kinase inhibitor (TKI), a poly (ADP-ribose) polymerase (PARP) inhibitor, an epidermal growth factor receptor (EGFR) inhibitor, an inosine monophosphate dehydrogenase (IMPDH) inhibitor, a rituximab monoclonal antibody against T and/or B cells, chemotherapy, total body radiation and localized radiation.

11. (canceled)

12. (canceled)

13. (canceled)

14. (canceled)

15. A method of screening an inhibitor of PIK3CA comprising i) providing a test compound and ii) determining the ability of said test compound to inhibit and/or reduce the activity and/or expression of PIK3CA.

16. The method of claim 9, wherein the PIK3CA inhibitor and the classical treatment are administered simultaneously in a combined preparation, or separately, or sequentially.
